(19) United States
(12) Patent Application Publication
(10) Pub. No.: US 2026/0232206 A1
(43) Pub. Date: Aug. 13, 2026

(54) PHYSIOLOGICAL INFORMATION PROCESSING APPARATUS, PHYSIOLOGICAL INFORMATION PROCESSING METHOD, AND PHYSIOLOGICAL INFORMATION PROCESSING PROGRAM

(71) Applicant: NIHON KOHDEN CORPORATION, Tokyo (JP)

(72) Inventors: Yoshihiro SUGO, Saitama (JP); Yoshiharu HARADA, Saitama (JP); Masumi KUBOTA, Saitama (JP); Sayaka YAMAZAKI, Saitama (JP)

(73) Assignee: NIHON KOHDEN CORPORATION, Tokyo (JP)

(21) Appl. No.: 19/153,125

(22) PCT Filed: Feb. 7, 2024
(86) PCT No.: PCT/JP2024/004118

§ 371 Date: Aug. 1, 2025

(30) Foreign Application Priority Data
Feb. 17, 2023 (JP) 2023-023465

(51) Int. Cl. A61B 5/021; A61B 5/352

(52) U.S. Cl. CPC A61B 5/02108; A61B 5/352

(57) ABSTRACT

A physiological information processing apparatus includes an ECG data detector configured to detect ECG data of a subject, a pulse wave data detector configured to detect pulse wave data of the subject, and a pulse wave transit time measurement unit configured to measure pulse wave transit time of the subject, based on the ECG data and the pulse wave data, The pulse wave transit time measurement unit includes a candidate value measurement unit configured to set one of rising timings of pulse waves as reference time, and configured to measure, as candidate values of the pulse wave transit time, time from the heartbeats to the reference time for a plurality of heartbeats before the reference time, and a specifying unit configured to specify, as the pulse wave transit time of the subject, the candidate value satisfying a prescribed condition, among the candidate values.

### TECHNICAL FIELD

The present disclosure relates to a physiological information processing apparatus, a physiological information processing method, and a physiological information processing program.

### BACKGROUND

JP2005-312947A discloses a technique of measuring pulse wave transit time (PWTT) of a subject to obtain cardiac output (CO) of the subject. [Summary]

### Technical Problem

However, when the subject's heart rate t is high, an interval between heartbeats (RR interval) narrows, and a pulse wave may rise after a heartbeat next to a heartbeat that generated the pulse wave. The same or similarly, when a pulse wave sensor is attached to a location away from the heart, the pulse wave may rise after the heartbeat next to the heartbeat that generated the pulse wave.

A measurement apparatus described in JP2005-312947A is based on a premise that the pulse wave rises from the immediately preceding heartbeat. Therefore, a technique capable of measuring more accurate PWTT is desired.

### Solution to Problem

A physiological information processing apparatus including:
an ECG data detector configured to detect ECG data of a subject;
a pulse wave data detector configured to detect pulse wave data of the subject; and
a pulse wave transit time measurement unit configured to measure pulse wave transit time of the subject, based on the ECG data and the pulse wave data,
in which the pulse wave transit time measurement unit includes:
a candidate value measurement unit configured to set one of rising timings of pulse waves as reference time, and configured to measure, as candidate values of the pulse wave transit time, time from the heartbeats to the reference time for a plurality of heartbeats before the reference time; and
a specifying unit configured to specify, as the pulse wave transit time of the subject, the candidate value satisfying a prescribed condition, among the candidate values.

A physiological information processing apparatus including:
an ECG data detector configured to detect ECG data of a subject;
a pulse wave data detector configured to detect pulse wave data of the subject; and
a pulse wave transit time measurement unit configured to measure pulse wave transit time of the subject, based on the ECG data and the pulse wave data,
in which the pulse wave transit time measurement unit includes:

a first candidate value measurement unit configured to set one of a plurality of heartbeats as reference time, and configured to measure time, as a first candidate value, from the reference time to a rising timing of a pulse wave after and closest to the reference time;
a second candidate value measurement unit configured to measure time, as a second candidate value, from the reference time to a rising timing of a pulse wave next to the pulse wave after and closest to the reference time;
a preceding RR interval measurement unit configured to measure a preceding RR interval that is time from a heartbeat before and closest to the reference time to the reference time;
an RR interval measurement unit configured to measure an RR interval that is time from the reference time to a heartbeat after and closest to the reference time; and
a specifying unit configured to specify the pulse wave transit time of the subject, based on the first candidate value, the second candidate value, the preceding RR interval, and the RR interval.

A physiological information processing method for a physiological information processing apparatus, the physiological information processing method including:
detecting ECG data of a subject;
detecting pulse wave data of the subject; and
measuring pulse wave transit time of the subject, based on the ECG data and the pulse wave data,
in which the measuring of the pulse wave transit time of the subject includes:
setting one of rising timings of pulse waves as reference time, and measuring, as candidate values of the pulse wave transit time, time from the heartbeats to the reference time for a plurality of heartbeats before the reference time; and
specifying, as the pulse wave transit time of the subject, the candidate value satisfying a prescribed condition among the candidate values.

A physiological information processing method for a physiological information processing apparatus, the physiological information processing method including:
detecting ECG data of a subject;
detecting pulse wave data of the subject; and
measuring pulse wave transit time of the subject, based on the ECG data and the pulse wave data,
in which the measuring of the pulse wave transit time of the subject includes:

setting one of a plurality of heartbeats as reference time and measuring time, as a first candidate value, from the reference time to a rising timing of a pulse wave after and closest to the reference time;
measuring time, as a second candidate value, from the reference time to a rising timing of a pulse wave next to the pulse wave after and closest to the reference time;
measuring a preceding RR interval that is time from a heartbeat before and closest to the reference time to the reference time;
measuring an RR interval that is time from the reference time to a heartbeat after and closest to the reference time; and
specifying the pulse wave transit time of the subject, based on the first candidate value, the second candidate value, the preceding RR interval, and the RR interval.

A physiological information processing program used for a physiological information processing apparatus, the program including instructions which, when executed by a computer, cause the computer in the physiological information processing apparatus to:
detect ECG data of a subject;
detect pulse wave data of the subject; and
measure pulse wave transit time of the subject, based on the ECG data and the pulse wave data,
in which the measuring of the pulse wave transit time of the subject includes:
setting one of rising timings of pulse waves as reference time, and measuring, as candidate values of the pulse wave transit time, time from the heartbeats to the reference time for a plurality of heartbeats before the reference time; and
specifying, as the pulse wave transit time of the subject, the candidate value satisfying a prescribed condition among the candidate values.

A physiological information processing program used for a physiological information processing apparatus, the program including instructions which, when executed by a computer, cause the computer in the physiological information processing apparatus to:
detect ECG data of a subject;
detect pulse wave data of the subject; and
measure pulse wave transit time of the subject, based on the ECG data and the pulse wave data,
in which the measuring of the pulse wave transit time of the subject includes:
setting one of a plurality of heartbeats as reference time and measuring time, as a first candidate value, from the reference time to a rising timing of a pulse wave after and closest to the reference time;
measuring time, as a second candidate value, from the reference time to a rising timing of a pulse wave next to the pulse wave after and closest to the reference time;
measuring a preceding RR interval that is time from a heartbeat before and closest to the reference time to the reference time;
measuring an RR interval that is time from the reference time to a heartbeat after and closest to the reference time; and
specifying the pulse wave transit time of the subject, based on the first candidate value, the second candidate value, the preceding RR interval, and the RR interval.

### Effects of the Invention

According to the present disclosure, pulse wave transit time of a subject can be measured more accurately.

### BRIEF DESCRIPTION OF DRAWINGS

FIG. 1 illustrates a configuration of a physiological information processing system 1 including a physiological information processing apparatus according to a first aspect of the present disclosure;

FIG. 2 illustrates a method for measuring pulse wave transit time by a pulse wave transit time measurement unit of FIG. 1;

FIG. 3 is a flowchart illustrating operation in a case where a physiological information processing apparatus according to a first embodiment specifies pulse wave transit time of a subject;

FIG. 4 illustrates a configuration of a physiological information processing apparatus according to a second aspect of the present disclosure;

FIG. 5 is a graph illustrating an example of values of a first correlation coefficient calculated by a first correlation coefficient calculator of FIG. 4, and values of a second correlation coefficient calculated by a second correlation coefficient calculator of FIG. 4;

FIG. 6 is a graph illustrating another example of values of the first correlation coefficient calculated by the first correlation coefficient calculator of FIG. 4, and values of the second correlation coefficient calculated by the second correlation coefficient calculator of FIG. 4;

FIG. 7 is a graph illustrating another example of values of the first correlation coefficient calculated by the first correlation coefficient calculator of FIG. 4, and values of the second correlation coefficient calculated by the second correlation coefficient calculator of FIG. 4;

FIG. 8 is a flowchart illustrating operation in a case where a physiological information processing apparatus according to a second embodiment specifies pulse wave transit time of a subject;

FIG. 9 illustrates a configuration of a physiological information processing apparatus according to a third aspect of the present disclosure;

FIG. 10 illustrates a method for measuring pulse wave transit time by a pulse wave transit time measurement unit of FIG. 9;

FIG. 11 is a graph illustrating an example of a first correlation coefficient calculated by a first calculator of FIG. 10;

FIG. 12 is a graph illustrating an example of a second correlation coefficient calculated by a second calculator of FIG. 10;

FIG. 13 illustrates an example of a relationship between a slope and a correlation coefficient calculated by the first calculator of FIG. 9, and a relationship between a slope and a correlation coefficient calculated by the second calculator of FIG. 9;

FIG. 14 is a flowchart illustrating operations in a case where a physiological information processing apparatus according to a third embodiment specifies pulse wave transit time of a subject;

FIG. 15 illustrates a case where a plurality of first candidate values measured by a first candidate value measurement unit of FIG. 9 are divided into a plurality of ranges;

FIG. 16 is a graph illustrating a relationship between a first candidate value in a case where a first candidate value group 1a of FIG. 15 is adopted, and a first candidate value in a case where a first candidate value group 1b of FIG. 15 is adopted;

FIG. 17 illustrates a case where a plurality of second candidate values measured by a second candidate value measurement unit of FIG. 9 are divided into a plurality of ranges;

FIG. 18 is a graph illustrating a relationship between a second candidate value in a case where a second candidate value group 2a of FIG. 17 is adopted, and a second candidate value in a case where a second candidate value group 2b of FIG. 17 is adopted; and

FIG. 19 illustrates an example of a relationship between slopes x0a and x0b and correlation coefficients y0a and y0b calculated by the first calculator of FIG. 9, and a relationship between slopes x1a and x1b and correlation coefficients y1a and y1b calculated by the second calculator of FIG. 9.

### DESCRIPTION OF EMBODIMENTS

Exemplary embodiments of a physiological information processing apparatus, a physiological information processing method, and a physiological information processing program according to the present disclosure will be described with reference to the accompanying drawings.

### First Embodiment

Configuration of Physiological information Processing System

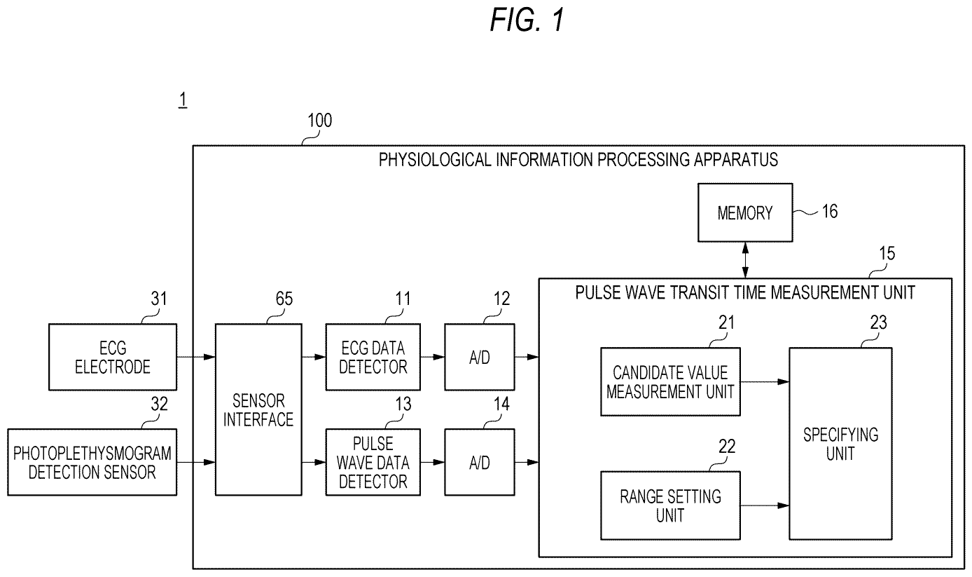

FIG. 1 illustrates a configuration of a physiological information processing system 1 including a physiological information processing apparatus 100 according to a first aspect of the present disclosure. As illustrated in FIG. 1, the physiological information processing system 1 can include the physiological information processing apparatus 100, an ECG electrode 31, and a photoplethysmogram detection sensor 32.

The ECG electrode 31 is attached to, for example, the subject's chest. The ECG electrode 31 is configured to measure a lead waveform as an ECG signal. The ECG electrode 31 is electrically connected to the physiological information processing apparatus 100. Measurement data by the ECG electrode 31 is input to the physiological information processing apparatus 100.

The photoplethysmogram detection sensor 32 is attached to a subject's periphery such as a finger. The sensor 32 is configured to measure a pulse wave of the subject. The sensor 32 is electrically connected to the physiological information processing apparatus 100. Measurement data from the sensor 32 is input to the physiological information processing apparatus 100.

The physiological information processing apparatus 100 can include an ECG data detector 11, an A/D converter 12, a pulse wave data detector 13, an A/D converter 14, a pulse wave transit time measurement unit 15, one or more memories 16, and a sensor interface 65.

The ECG data detector 11 is configured to detect the subject's ECG data, based on the measurement data received from the ECG electrode 31 via the sensor interface 65. The ECG data detector 11 is configured to output an analog signal indicating a detected ECG waveform to the A/D converter 14. The A/D converter 14 is configured to convert the analog signal output from the ECG data detector 11 into a digital signal, and configured to output the digital signal to the pulse wave transit time measurement unit (PWTT measurement unit) 15.

The pulse wave data detector 13 is configured to detect pulse wave data in the subject's periphery, based on the measurement data received from the photoplethysmogram detection sensor 32 via the sensor interface 65. The pulse wave data detector 13 is configured to output, to the A/D converter 14, an analog signal indicating a waveform of detected photoplethysmogram of the peripheral part. The A/D converter 14 is configured to convert the analog signal output from the pulse wave data detector 13 into a digital signal, and configured to output the digital signal to the PWTT measurement unit 15.

The PWTT measurement unit 15 is configured to measure the pulse wave transit time (PWTT) of the subject, based on the ECG data detected by the ECG electrode 31 and the pulse wave data detected by the photoplethysmogram detection sensor 32.

The one or more memories 16 is configured to store attribute information on the subject. The attribute information indicates at least one of parameters such as age, gender, height, weight, albumin value, glucose value, hemoglobin value, urea nitrogen value, and estimated blood vessel age of the subject. The attribute information is input in advance by, for example, an operator who manages physiological information on the subject.

The physiological information processing system 1 can include a central processing unit (CPU), a read only memory (ROM), a random-access memory (RAM), a hard disk drive (HDD), and the like. The CPU may function as the ECG data detector 11, the pulse wave data detector 13, the PWTT measurement unit 15, and the like.

### Details of Pulse Wave Transit Time Measurement Unit

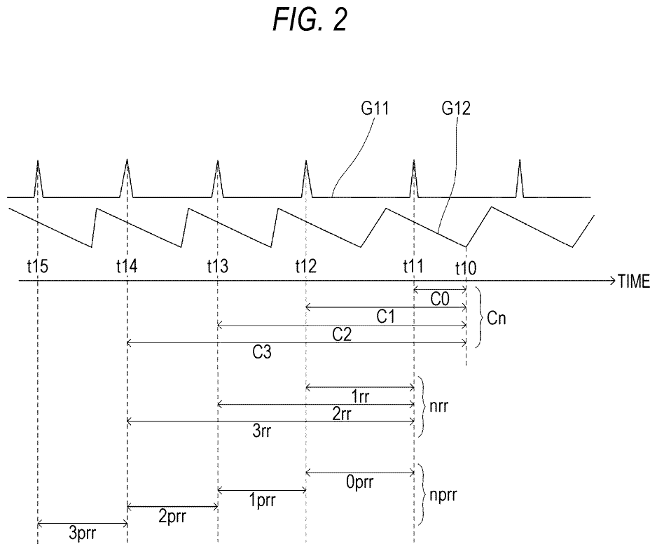

FIG. 2 illustrates a method for measuring the PWTT by the PWTT measurement unit 15 of FIG. 1. A graph G11 of FIG. 2 illustrates an ECG waveform based on ECG data, in which a horizontal axis represents time. A graph G12 of FIG. 2 illustrates a waveform of the photoplethysmogram based on the pulse wave data, in which the horizontal axis indicates time.

Referring to FIGS. 1 and 2, the PWTT measurement unit 15 can include a candidate value measurement unit 21, a range setting unit 22, and a specifying unit 23. The candidate value measurement unit 21 is configured to measure a plurality of candidate values Cn (n is zero or a natural number) of the PWTT, based on the ECG data and the pulse wave data.

More specifically, as illustrated in FIG. 2, the candidate value measurement unit 21 is configured to set, as reference time t10, one of rising timings of pulse waves. The candidate value measurement unit 21 is configured to measure, as the candidate values Cn of the PWTT, time from R wave generation time points (hereinafter referred to as “R timings”) to the reference time t10, for a plurality of heartbeats before the reference time t10.

Specifically, among the R timings, the R timing before and closest to the reference time t10 is set as a time point t11. In addition, the R timing immediately before the time point t11 is t12, the R timing immediately before the time point t12 is t13, and the R timing immediately before the time point t13 is t14. In this case, the candidate value measurement unit 21 is configured to set time from the time point t11 to the reference time t10 as a candidate value C0, sets time from the time point t12 to the reference time t10 as a candidate value C1, sets time from the time point t13 to the reference time t10 as a candidate value C2, and sets time from the time point t14 to the reference time t10 as a candidate value C3. The candidate value measurement unit 21 is configured to output, to the specifying unit 23, the plurality of measured candidate values Cn.

The range setting unit 22 is configured to set, based on the attribute information stored in the one or more memories 16, a standard range in which the PWTT of the subject is included. For example, the range setting unit 22 is configured to set the standard range by performing regression analysis using a plurality of parameters indicated by the attribute information. The range setting unit 22 is configured to output the set standard range to the specifying unit 23.

The range setting unit 22 may be configured to set the standard range by machine learning other than regression analysis. The range setting unit 22 is not limited to setting the standard range, and may be configured to set, for example, only a lower limit value of the standard range or only an upper limit value of the standard range. The standard range may be a preset fixed value.

The specifying unit 23 is configured to specify, as the PWTT of the subject, the candidate value Cn satisfying a prescribed condition among the plurality of candidate values Cn measured by the candidate value measurement unit 21. In this example, the specifying unit 23 is configured to specify, as the PWTT of the subject, the smallest candidate value Cn that is equal to or greater than the lower limit value of the standard range set by the range setting unit 22, among the plurality of candidate values C.

Specifically, it is assumed that the candidate value C1, the candidate value C2, the candidate value C3 . . . of the plurality of candidate values Cn are equal to or greater than the lower limit value of the standard range. In this case, the specifying unit 23 specifies, as the PWTT of the subject, the smallest candidate value C1 among the candidate values Cn. The specifying unit 23 then outputs the specified PWTT to, for example, an information processing unit (not illustrated).

### Operations

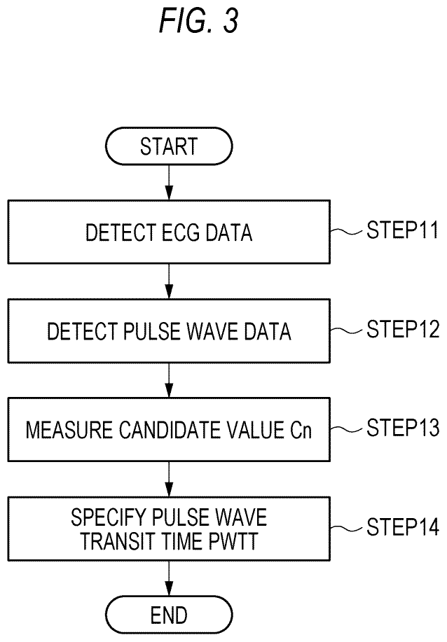

FIG. 3 is a flowchart illustrating operations in a case where the physiological information processing apparatus 100 according to a first embodiment specifies the PWTT of the subject.

Referring to FIG. 3, first, the physiological information processing apparatus 100 detects the ECG data of the subject, based on the measurement data received from the ECG electrode 31 (STEP 11).

Next, the physiological information processing apparatus 100 detects the pulse wave data of the peripheral part of the subject, based on the measurement data received from the photoplethysmogram detection sensor 32 (STEP 12).

Next, the physiological information processing apparatus 100 measures, as the candidate value Cn, time from the R timing of the heartbeat to the reference time t10, for each of a plurality of heartbeats before the reference time t10 that is one of rising timings of the pulse waves (STEP 13).

Next, the physiological information processing apparatus 100 specifies, as the PWTT of the subject, the candidate value Cn satisfying the prescribed condition among the plurality of candidate values Cn. Specifically, the physiological information processing apparatus 100 specifies, as the PWTT of the subject, the smallest candidate value Cn that is equal to or greater than the lower limit value of the standard range set by the range setting unit 22, among the plurality of candidate values C (STEP 14). With this operation, the PWTT of the subject can be measured more accurately.

### Modifications of First Embodiment

Referring again to FIG. 1, the specifying unit 23 may be configured to specify, as the PWTT of the subject, the candidate value Cn that is equal to or greater than the lower limit value of the standard range and less than the upper limit value of the standard range, among the plurality of candidate values Cn.

Specifically, it is assumed that, among the plurality of candidate values Cn in FIG. 2, the candidate value Cn that is equal to or greater than the lower limit value of the standard range and less than the upper limit value of the standard range is the candidate value C2. In this case, the specifying unit 23 specifies the candidate value C2 as the PWTT of the subject.

In a case where there are two or more candidate values Cn that are greater than or equal to the lower limit value of the standard range and less than the upper limit value of the standard range among the plurality of candidate values Cn, the specifying unit 23 specifies, for example, the minimum value among the two or more candidate values Cn, as the PWTT of the subject. Specifically, it is assumed that the candidate value C2 and the candidate value C3 are equal to or greater than the lower limit value of the standard range and less than the upper limit value of the standard range. In this case, the specifying unit 23 specifies the candidate value C2 as the PWTT of the subject.

As described above, in the physiological information processing apparatus 100 according to the first aspect of the present disclosure, the ECG data detector 11 is configured to detect the ECG data of the subject. The pulse wave data detector 13 is configured to detect the pulse wave data of the subject. The PWTT measurement unit 15 is configured to measure the PWTT of the subject, based on the ECG data and the pulse wave data. In the PWTT measurement unit 15, the candidate value measurement unit 21 is configured to set one of the rising timings of the pulse waves as the reference time t10, and configured to measure, as the candidate value Cn of the PWTT, the time from the heartbeat to the reference time t10, for each of a plurality of heartbeats before the reference time t10. In the PWTT measurement unit 15, the specifying unit 23 is configured to specify, as the PWTT of the subject, the candidate value Cn satisfying the prescribed condition among the plurality of candidate values Cn.

As described above, a measuring apparatus described in JP2005-312947A is based on a premise that a pulse wave rises from an immediately preceding heartbeat. In JP2020-39384A, accurate PWTT of a subject is measured by a respiratory variation of the PWTT within prescribed time. However, the respiratory variation of the PWTT depends on a respiratory state such as ventilation amount and intrathoracic pressure, and thus it is difficult to measure the accurate PWTT in a case where the respiratory state greatly varies.

On the other hand, the PWTT of the subject can be more accurately measured by measuring a plurality of candidate values Cn of the PWTT, and by specifying the candidate value satisfying a prescribed condition from the plurality of candidate values Cn.

In the physiological information processing apparatus 100 according to other aspect of the present disclosure, the specifying unit 23 is configured to specify, as the PWTT of the subject, the smallest candidate value Cn that is equal to or greater than the lower limit value of the set standard range of the PWTT.

With such a configuration, it is possible to easily measure the more accurate PWTT of the subject without performing complicated processing.

In the physiological information processing apparatus 100 according to other aspect of the present disclosure, the specifying unit 23 is configured to specify, as the PWTT of the subject, the candidate value Cn that is equal to or greater than the lower limit value of the set standard range of the PWTT and less than the upper limit value of the standard range, among the plurality of candidate values Cn.

With such a configuration, it is possible to easily measure the more accurate PWTT of the subject without performing complicated processing.

As described above, in the physiological information processing apparatus 100 according to the other of the present disclosure, the range setting unit 22 of the PWTT measurement unit sets the standard range, based on attributes of the subject.

In this manner, setting the standard range of the PWTT in accordance with the attributes of the subject makes it possible to measure the PWTT more accurately.

### Second Embodiment

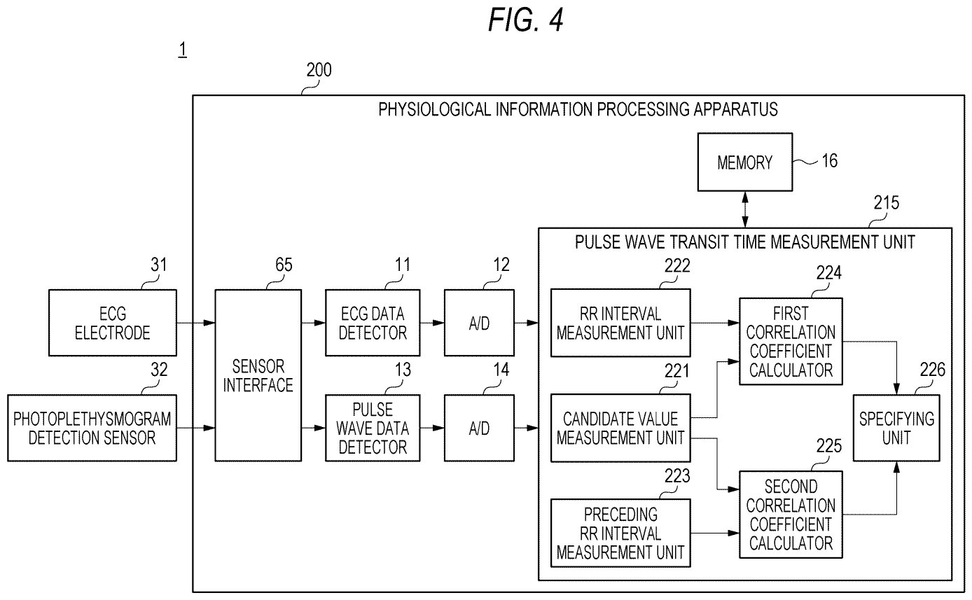

Next, a physiological information processing apparatus 200 according to a second aspect of the present disclosure will be described. FIG. 4 illustrates a configuration of the physiological information processing apparatus 200 according to the second aspect of the present disclosure. As illustrated in FIG. 4, the physiological information processing apparatus 200 is different from the physiological information processing apparatus 100 in FIG. 1 in that the physiological information processing apparatus 200 can include a PWTT measurement unit 215, instead of the PWTT measurement unit 15. Other configurations of the physiological information processing apparatus 200 are the same as or similar to those of the physiological information processing apparatus 100 in FIG. 1, and thus detailed description thereof will not be repeated here.

### Configuration of Pulse Wave Transit Time Measurement Unit

The PWTT measurement unit 215 is configured to specify PWTT of a subject, based on a correlation between the candidate value Cn and an RR interval that is an interval between a plurality of R timings, and a correlation between the candidate value Cn and a preceding RR interval that is an interval between successive R timings.

More specifically, referring to FIGS. 2 and 4, the PWTT measurement unit 215 can include a candidate value measurement unit 221, an RR interval measurement unit 222, a preceding RR interval measurement unit 223, a first correlation coefficient calculator 224, a second correlation coefficient calculator 225, and a specifying unit 226. The same as or similar to the candidate value measurement unit 21 of FIG. 1, the candidate value measurement unit 221 is configured to measure a plurality of candidate values Cn of the PWTT, based on ECG data and pulse wave data.

The RR interval measurement unit 222 is configured to measure, as an RR interval nrr, time from each heartbeat before the reference time t10 to the time point t11 that is the R timing of the heartbeat before and closest to the reference time t10. Specifically, the RR interval measurement unit 222 is configured to measure time from the time point t12 to the time point t11 as an RR interval 1rr, is configured to measure time from the time point t13 to the time point t11 as an RR interval 2rr, and is configured to measure time from the time point t14 to the time point t11 as an RR interval 3rr.

The preceding RR interval measurement unit 223 is configured to measure a preceding RR interval nprr that is time from the R timing of an immediately preceding heartbeat to each heartbeat before the reference time t10. Specifically, the preceding RR interval measurement unit 223 is configured to measure time from the time point t12 to the time point t11 as a preceding RR interval 0prr, is configured to measure time from the time point t13 to the time point t12 as a preceding RR interval 1prr, and is configured to measure time from the time point t14 to the time point t13 as a preceding RR interval 2prr.

The first correlation coefficient calculator 224 is configured to calculate a first correlation coefficient Ri (i is zero or a natural number) between the candidate value Cn measured by the candidate value measurement unit 221 and the RR interval nrr measured by the RR interval measurement unit 222.

Specifically, the first correlation coefficient calculator 224 is configured to calculate, as a first correlation coefficient R1, a first correlation coefficient Ri between the candidate value C1 and the RR interval 1rr. The first correlation coefficient calculator 224 is configured to calculate, as a first correlation coefficient R2, the first correlation coefficient Ri between the candidate value C2 and the RR interval 2rr. The first correlation coefficient calculator 224 is configured to calculate, as a first correlation coefficient R3, the first correlation coefficient Ri between the candidate value C3 and the RR interval 3rr.

The second correlation coefficient calculator 225 is configured to calculate a second correlation coefficient pRi between the candidate value Cn measured by the candidate value measurement unit 221 and the preceding RR interval nprr measured by the preceding RR interval measurement unit 223.

Specifically, the second correlation coefficient calculator 225 is configured to calculate, as a second correlation coefficient pR0, the second correlation coefficient pRi between the candidate value C0 and the preceding RR interval 0prr. The second correlation coefficient calculator 225 is configured to calculate, as a second correlation coefficient pR1, the second correlation coefficient pRi between the candidate value C1 and the preceding RR interval 1prr. The second correlation coefficient calculator 225 is configured to calculate, as a second correlation coefficient pR2, the second correlation coefficient pRi between the candidate value C2 and the preceding RR interval 2prr. The second correlation coefficient calculator 225 is configured to calculate, as a second correlation coefficient pR3, the second correlation coefficient pRi between the candidate value C3 and the preceding RR interval 3prr.

The specifying unit 226 is configured to specify the PWTT of the subject, based on the correlation between the candidate value Cn and the RR interval nrr and based on the correlation between the candidate value Cn and the preceding RR interval nprr. That is, the specifying unit 226 is configured to specify the PWTT of the subject, based on the first correlation coefficient Ri calculated by the first correlation coefficient calculator 224 and the second correlation coefficient pRi calculated by the second correlation coefficient calculator 225.

### Details of Method for Specifying Pulse Wave Transit Time by Specifying Unit

(Relationship Between Candidate Value Cn and RR Interval nrr)

Here, referring to FIG. 2, it is assumed that a pulse wave rises at the reference time t10 in accordance with heart pumping corresponding to the time point t13. That is, it is assumed that the candidate value C2 is the accurate PWTT.

In this case, the candidate value C2 is an independent value having no correlation with time related to a time point later than the time point t13. That is, there is no correlation between the candidate value C2 and the RR interval 2rr. On the other hand, there is a positive correlation between the candidate value C3 and the RR interval 3rr as derived from the following Equations (1) and (2).

That is, the candidate value C3 satisfies the following equation (1).

$$C 3 = C 2 + ( 3 rr - 2 rr ) \qquad ( 1 )$$

The RR interval 3rr satisfies the following equation (2).

$$3 rr = 2 rr + ( 3 rr - 2 rr ) \qquad ( 2 )$$

Since the candidate value C2 is an independent value as described above, it can be seen from Equation (1) that there is a positive correlation between the candidate value C3 and (RR interval 3rr-RR interval 2rr). Further, it can be seen from Equation (2) that there is a positive correlation between the RR interval 3rr and (RR interval 3rr-RR interval 2rr). Accordingly, it can be seen that there is a positive correlation between the candidate value C3 and the RR interval 3rr.

The same as or similar to the candidate value C3, the candidate values C4, C5, . . . have a positive correlation with the RR intervals 4rr, 5rr, . . . , respectively.

In this manner, in a case where the candidate value C2 is the accurate PWTT, there is no correlation between the candidate value C2 and the RR interval 2rr. On the other hand, in this case, there is a positive correlation between the candidate values C3, C4, . . . and the RR intervals 3rr, 4rr, . . . , respectively.

For this reason, for example, the specifying unit 226 is configured to determine whether the first correlation coefficient Ri calculated by the first correlation coefficient calculator 224 is equal to or greater than a first threshold Th1 used to specify the PWTT, by setting the first threshold Th1 to zero.

### (Relationship Between Candidate Value Cn and Preceding RR Interval Nprr)

Here, it is assumed that the candidate value C2 is the accurate PWTT. In this case, the candidate value C2 depends on the amount of blood stored in the heart during time from the time point t14 to the time point t13. For this reason, there is a negative correlation between the candidate value C2 and the preceding RR interval 2prr.

Further, there is a negative correlation between the candidate value C1 and the preceding RR interval 1prr as derived from the following Equation (3). That is, the candidate values C1 and C2 satisfy the following Equation (3).

$$C 2 = 1 prr + C 1 \qquad ( 3 )$$

Since the candidate value C2 is an independent value as described above, it can be seen that there is a negative correlation between the candidate value C1 and the preceding RR interval 1prr. The same as or similar to the candidate value C1, the candidate value C0 has a negative correlation with the preceding RR interval 0rr.

In this manner, in a case where the candidate value C2 is the accurate PWTT, the candidate values C0, C1, and C2 have a negative correlation with the preceding RR intervals 0prr, 1prr, and 2prr, respectively. On the other hand, in this case, there is no correlation between the candidate values C3, C4, . . . and the preceding RR intervals 3prr, 4prr, . . . , respectively.

Therefore, for example, the specifying unit 226 is configured to determine whether the second correlation coefficient pRi calculated by the second correlation coefficient calculator 225 is equal to or greater than a second threshold Th2 used to specify the PWTT, by setting the second threshold Th2 to zero.

The first threshold Th1 and the second threshold Th2 are not limited to zero. For example, the first threshold Th1 may be a value larger than zero and smaller than 1(0<Th1<1). For example, the second threshold Th2 may be a value larger than −1 and smaller than zero (−1<Th2<0).

The specifying unit 226 may be configured to specify the PWTT using one of the first correlation coefficient Ri and the second correlation coefficient pRi. For example, the specifying unit 226 may be configured to specify the PWTT by determining whether the first correlation coefficient Ri is equal to or greater than the first threshold Th1. In this case, the PWTT measurement unit 215 may not include the preceding RR interval measurement unit 223 and the second correlation coefficient calculator 225.

For example, the specifying unit 226 may be configured to specify the PWTT by determining whether the second correlation coefficient pRi is equal to or greater than the second threshold Th2. In this case, the PWTT measurement unit 215 may not include the RR interval measurement unit 222 and the first correlation coefficient calculator 224.

However, in a case where both the first correlation coefficient Ri and the second correlation coefficient pRi are used, the PWTT can be more accurately specified. For this reason, the specifying unit 226 is preferably configured to specify the PWTT using both the first correlation coefficient Ri and the second correlation coefficient pRi.

### Specific Example 1: In a Case Where Candidate Value C0 is Accurate PWTT

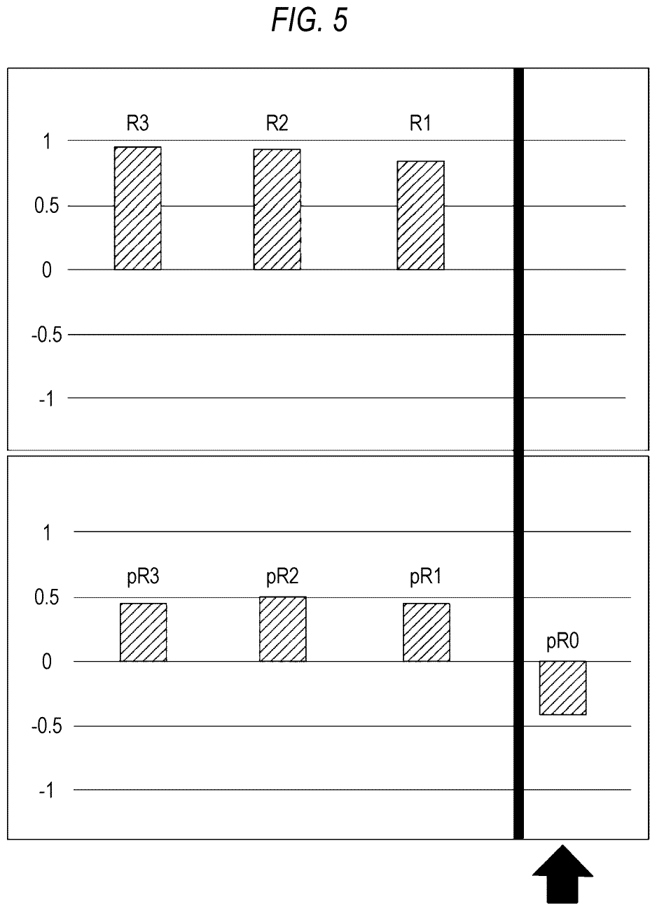

FIG. 5 is a graph illustrating an example of values of the first correlation coefficient Ri calculated by the first correlation coefficient calculator 224 of FIG. 4, and values of the second correlation coefficient pRi calculated by the second correlation coefficient calculator 225 of FIG. 4. FIG. 5 illustrates the first correlation coefficients R1, R2, and R3 and the second correlation coefficients pR0, pR1, pR2, and pR3.

In the example illustrated in FIG. 5, the first correlation coefficients R1, R2, and R3 are equal to or greater than the first threshold Th1. The second correlation coefficient pR0 is less than the second threshold Th2, and the second correlation coefficients pR1, pR2, and pR3 are equal to or greater than the second threshold Th2. In such a case, the specifying unit 226 is configured to specify the candidate value C0, as the accurate PWTT, among the plurality of candidate values Cn measured by the candidate value measurement unit 221.

### Specific Example 2: In a Case Where Candidate Value C1 is Accurate PWTT

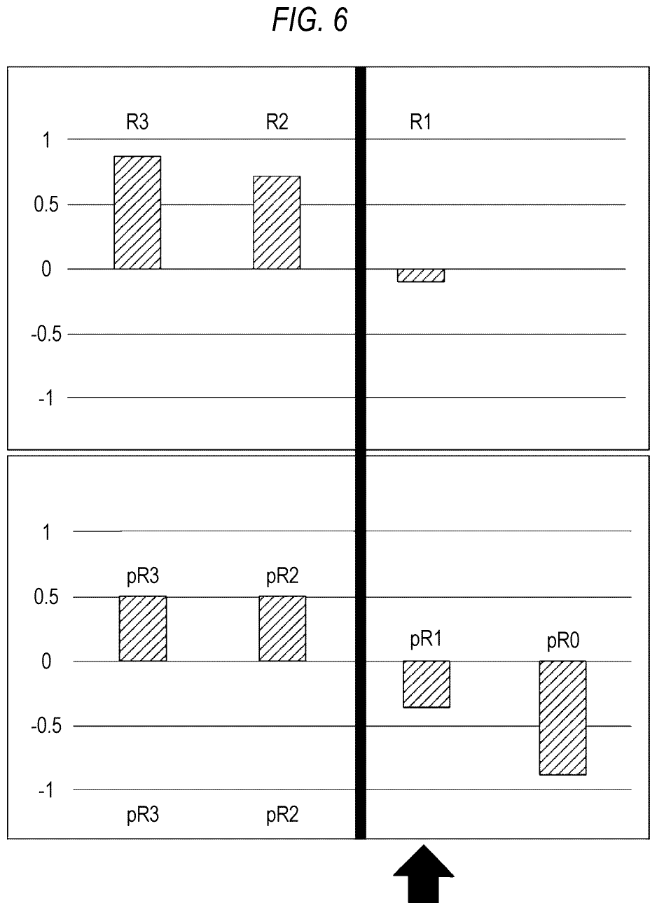

FIG. 6 illustrates another example of values of the first correlation coefficient Ri calculated by the first correlation coefficient calculator 224 of FIG. 4, and values of the second correlation coefficient pRi calculated by the second correlation coefficient calculator 225 of FIG. 4. The same as or similar to FIG. 5, FIG. 6 illustrates the first correlation coefficients R1, R2, and R3 and the second correlation coefficients pR0, pR1, pR2, and pR3.

In the example illustrated in FIG. 6, the first correlation coefficient R1 is less than the first threshold Th1, and the first correlation coefficients R2 and R3 are equal to or greater than the first threshold Th1. The second correlation coefficients pR0 and pR1 are less than the second threshold Th2, and the second correlation coefficients pR2 and pR3 are equal to or greater than the second threshold Th2. In such a case, the specifying unit 226 is configured to specify the candidate value C1, as the accurate PWTT, among the plurality of candidate values Cn measured by the candidate value measurement unit 221.

### Specific Example 3: In a Case Where Candidate Value C2 is Accurate PWTT

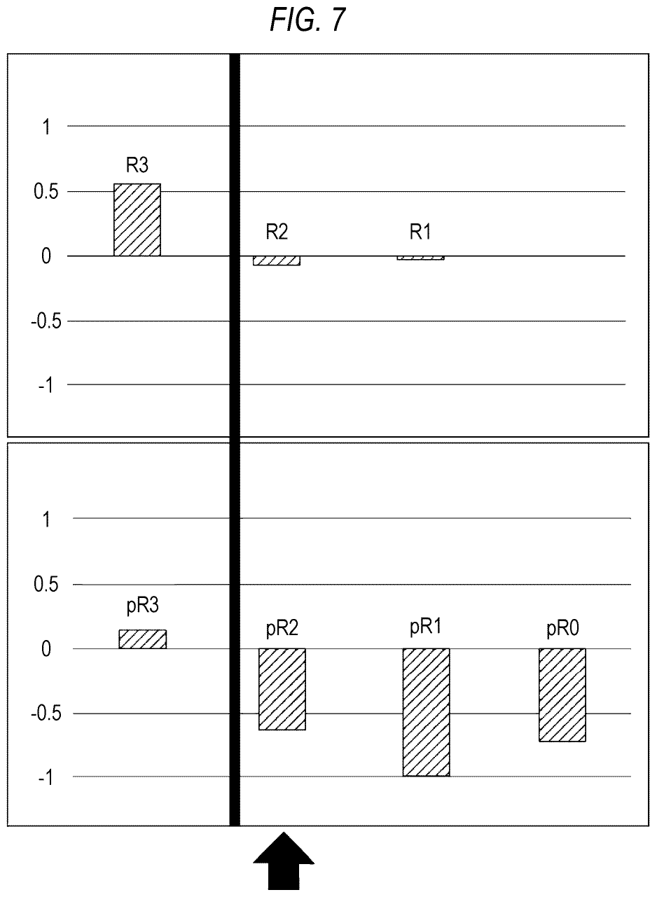

FIG. 7 illustrates another example of values of the first correlation coefficient Ri calculated by the first correlation coefficient calculator 224 of FIG. 4, and values of the second correlation coefficient pRi calculated by the second correlation coefficient calculator 225 of FIG. 4. The same as or similar to FIGS. 5 and 6, FIG. 7 illustrates the first correlation coefficients R1, R2, and R3 and the second correlation coefficients pR0, pR1, pR2, and pR3.

In the example illustrated in FIG. 7, the first correlation coefficients R1 and R² are less than the first threshold Th1, and the first correlation coefficient R3 is equal to or greater than the first threshold Th1. The second correlation coefficients pR0, pR1, and pR2 are less than the second threshold Th2, and the second correlation coefficient pR3 is equal to or greater than the second threshold Th2. In such a case, the specifying unit 226 is configured to specify the candidate value C2, as the accurate PWTT, among the plurality of candidate values Cn measured by the candidate value measurement unit 221.

### Operations

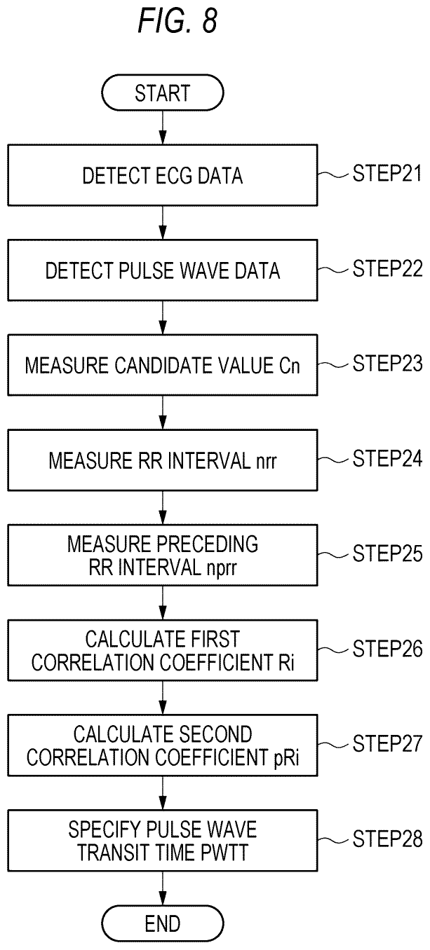

FIG. 8 is a flowchart illustrating operations in a case where the physiological information processing apparatus 200 according to the second embodiment specifies the PWTT of the subject.

Referring to FIG. 8, operation from STEP 21 to STEP 23 of FIG. 8 is the same as or similar to the operation from STEP 11 to STEP 13 of FIG. 3, and thus detailed description thereof will not be repeated here.

Next, the physiological information processing apparatus 200 measures the RR interval nrr that is an interval between a plurality of R timings (STEP 24).

Next, the physiological information processing apparatus 200 measures the preceding RR interval nprr that is an interval between successive R timings (STEP 25).

Next, the physiological information processing apparatus 200 calculates the first correlation coefficient Ri between the candidate value Cn and the RR interval nrr (STEP 26).

Next, the physiological information processing apparatus 200 calculates the second correlation coefficient pRi (STEP 27) between the candidate value Cn and the preceding RR interval nprr.

Next, the physiological information processing apparatus 200 specifies the PWTT of the subject, among the plurality of candidate values Cn, based on at least one of the first correlation coefficient Ri and the second correlation coefficient pRi (STEP 28).

For example, the physiological information processing apparatus 200 specifies a value k in which the first correlation coefficient Ri (i=k (k is a natural number)) is less than the first threshold Th1 and the first correlation coefficient Ri (i=k+1) is equal to or greater than the first threshold Th1, among the first correlation coefficients Ri. Then, the physiological information processing apparatus 200 specifies, as the PWTT of the subject, the candidate value Cn (n=k) among the plurality of candidate values Cn.

The physiological information processing apparatus 200 may specify, for example, the value k in which the second correlation coefficient pRi (i=k) is less than the second threshold Th2 and the second correlation coefficient pRi (i=k+1) is greater than or equal to the second threshold Th2, among the second correlation coefficients pRi. Also in this case, the physiological information processing apparatus 200 specifies, as the PWTT of the subject, the candidate value Cn (n=k) among the plurality of candidate values Cn.

As described above, in the physiological information processing apparatus 200 according to the second aspect of the present disclosure, the RR interval measurement unit 222 is configured to measure, as the RR interval nrr, each heartbeat before the reference time t10 to the heartbeat before and closest to the reference time t10. The specifying unit 226 is configured to specify the PWTT of the subject, based on the first correlation coefficient Ri between the candidate value Cn and the RR interval nrr.

Here, for example, it is assumed that the candidate value C2, which is time from the heartbeat two heartbeats before the heartbeat closest to the reference time t10 to the reference time t10, is the correct PWTT. In this case, there is no correlation between the candidate value C2 and the RR interval 2rr that is time from the heartbeat two heartbeats before the heartbeat closest to the reference time t10 to the heartbeat closest to the reference time t10.

On the other hand, in this case, there is a positive correlation between the RR interval 3rr that is time from the heartbeat three heartbeats before the heartbeat closest to the reference time t10 to the heartbeat closest to the reference time t10, and the candidate value C3 that is time from the heartbeat three heartbeats before the heartbeat closest to the reference time t10 to the reference time t10.

In this manner, the PWTT of the subject can be measured with higher accuracy by focusing on a change in the correlation between the candidate value Cn and the RR interval rr.

As described above, in the physiological information processing apparatus 200 according to the second aspect of the present disclosure, the preceding RR interval measurement unit 223 is configured to measure the preceding RR interval nrr that is the time from the immediately preceding heartbeat of each heartbeat before the reference time t10 to the each heartbeat before the reference time t10. The specifying unit 226 is configured to specify the PWTT of the subject, based on the second correlation coefficient pRi between the candidate value Cn and the preceding RR interval nrr.

Here, for example, it is assumed that the candidate value C2, which is time from the heartbeat two heartbeats before the heartbeat closest to the reference time t10 to the reference time t10, is the correct PWTT. In this case, there is a substantially negative correlation between the candidate value C2 and the preceding RR interval 2prr that is time from the heartbeat three heartbeats before the heartbeat closest to the reference time t10, to the heartbeat two heartbeats before the heartbeat closest to the reference time t10.

On the other hand, in this case, there is a positive correlation between the preceding RR interval 3prr that is time from the heartbeat four heartbeats before the heartbeat closest to the reference time t10 to the heartbeat three heartbeats before the heartbeat closest to the reference time t10, and the candidate value C3 that is time from the heartbeat three heartbeats before the heartbeat closest to the reference time t10 to the reference time t10.

Thus, the PWTT of the subject can be measured with higher accuracy by focusing on a change in the correlation between the candidate value Cn and the preceding RR interval rpp.

### Third Embodiment

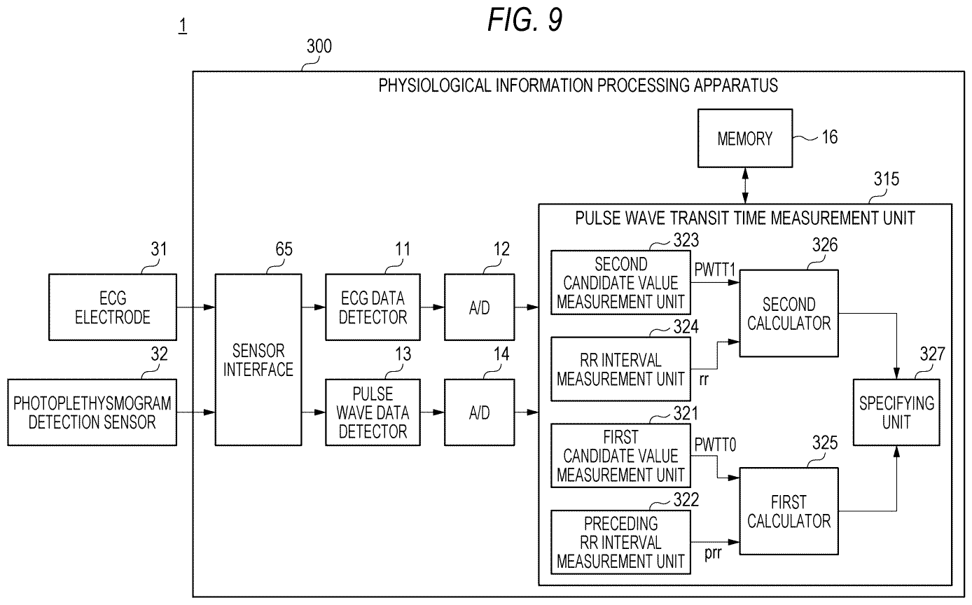

Next, a physiological information processing apparatus 300 according to a third aspect of the present disclosure will be described. FIG. 9 illustrates a configuration of the physiological information processing apparatus 300 according to the third aspect of the present disclosure.

As illustrated in FIG. 9, the physiological information processing apparatus 300 is different from the physiological information processing apparatus 100 in FIG. 1 in that the physiological information processing apparatus 300 can include a PWTT measurement unit 315, instead of the PWTT measurement unit 15. Other configurations of the physiological information processing apparatus 300 are the same as or similar to those of the physiological information processing apparatus 100 of FIG. 1, and thus detailed description thereof will not be repeated here.

### Configuration of Pulse Wave Transit Time Measurement Unit

The PWTT measurement unit 315 is configured to measure two types of candidate values, and is configured to specify one of the two types of candidate values as PWTT of a subject.

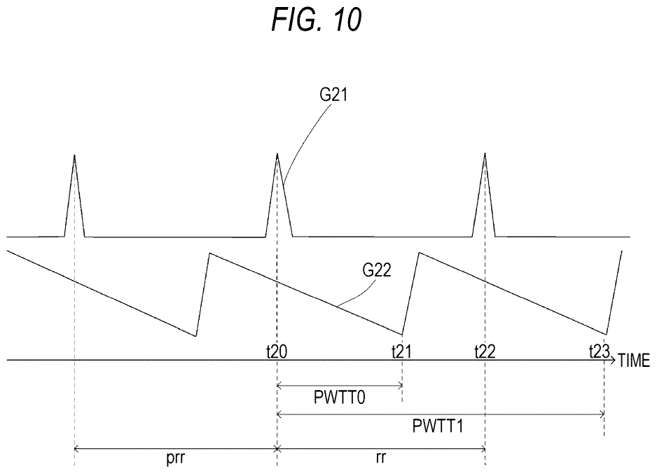

FIG. 10 illustrates a method for measuring the PWTT by the PWTT measurement unit 315 of FIG. 9. A graph G21 of FIG. 10 illustrates an ECG waveform based on ECG data, in which a horizontal axis represents time. A graph G22 in FIG. 10 illustrates a waveform of photoplethysmogram based on pulse wave data, in which the horizontal axis indicates time.

Referring to FIGS. 9 and 10, the PWTT measurement unit 315 can include a first candidate value measurement unit 321, a preceding RR interval measurement unit 322, a second candidate value measurement unit 323, an RR interval measurement unit 324, a first calculator 325, a second calculator 326, and a specifying unit 327.

The first candidate value measurement unit 321 is configured to set one of a plurality of heartbeats as a reference time t20, and is configured to measure, as a first candidate value PWTT0, time from the reference time t20 to a rising timing t21 of a pulse wave that is after and closest to the reference time t20. Then, the first candidate value measurement unit 321 is configured to output the measurement result to the first calculator 325.

The preceding RR interval measurement unit 322 is configured to measure the preceding RR interval prr that is time from a heartbeat before and closest to the reference time t20 to the reference time t20. Then, the preceding RR interval measurement unit 322 is configured to output the measurement result to the first calculator 325.

The second candidate value measurement unit 323 is configured to measure, as a second candidate value PTWW1, time from the reference time t20 to a rising timing t23 of a pulse wave that is after the reference time t20 and next to the pulse wave closest to the reference time t20. Then, the second candidate value measurement unit 323 is configured to output the measurement result to the second calculator 326.

The RR interval measurement unit 324 is configured to measure the RR interval rr that is time from the reference time t20 to the heartbeat that is after and closest to the reference time t20. Then, the RR interval measurement unit 324 is configured to output the measurement result to the second calculator 326.

The first calculator 325 is configured to calculate a first correlation between the first candidate value PWTT0 measured by the first candidate value measurement unit 321, and the preceding RR interval prr measured by the preceding RR interval measurement unit 322.

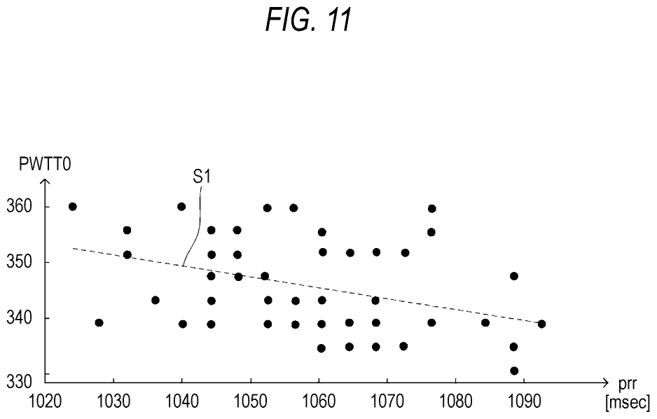

FIG. 11 is a graph illustrating an example of the first correlation coefficient calculated by the first calculator 325 of FIG. 10. In the graph in FIG. 11, a horizontal axis represents a value of the preceding RR interval prr, and a vertical axis represents a value of the first candidate value PWTT0.

For example, the first candidate value measurement unit 321 is configured to measure a plurality of first candidate values PWTT0, based on the ECG data and the pulse wave data obtained in one minute. The preceding RR interval measurement unit 322 is configured to measure a plurality of preceding RR intervals prr, based on the ECG data and the pulse wave data obtained in one minute. Then, the first calculator 325 is configured to calculate, as the first correlation, a slope x0 and a correlation coefficient y0 of a regression line S1 in a case where a plurality of points are plotted with the preceding RR interval prr being a value of the horizontal axis and the first candidate value PWTT0 being a value of the vertical axis.

Referring again to FIG. 10, the second calculator 326 is configured to calculate a second correlation between the second candidate value PWTT1 measured by the second candidate value measurement unit 323, and the RR interval rr measured by the RR interval measurement unit 324.

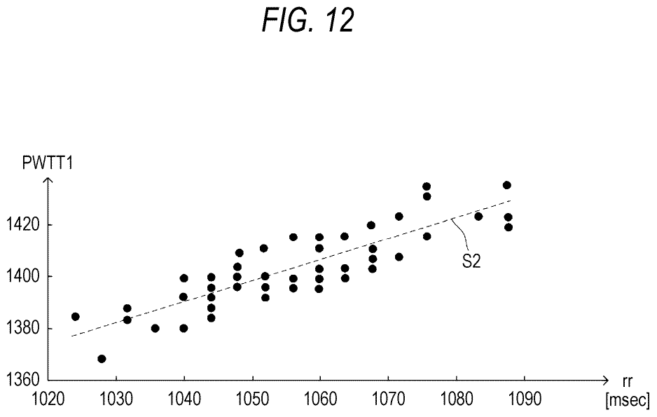

FIG. 12 is a graph of an example of a second correlation coefficient calculated by the second calculator 326 of FIG. 10. In the graph illustrated in FIG. 12, a horizontal axis represents a value of the RR interval rr, in which a vertical axis represents a value of the second candidate value PWTT1.

For example, the second candidate value measurement unit 323 is configured to measure a plurality of second candidate values PWTT1, based on the ECG data and the pulse wave data obtained in one minute. The RR interval measurement unit 324 is configured to measure a plurality of RR intervals rr, based on the ECG data and the pulse wave data obtained in one minute. Then, the second calculator 326 is configured to calculate, as the second correlation, a slope x1 and a correlation coefficient y1 of a regression line S2 in a case where a plurality of points are plotted with the RR interval rr being a value of the horizontal axis and the second candidate value PWTT1 being a value of the vertical axis.

Referring again to FIG. 9, the specifying unit 327 is configured to specify the accurate PWTT of the subject, based on the first candidate value PWTT0, the second candidate value PWTT1, the preceding RR interval prr, and the RR interval rr. That is, the specifying unit 327 is configured to specify, as the accurate PWTT of the subject, one of the first candidate value PWTT0 and the second candidate value PWTT1, based on the first correlation calculated by the first calculator 325 and the second correlation calculated by the second calculator 326.

### Details of Method for Specifying Pulse Wave Transit Time by Specifying Unit

### (Relationship Between First Candidate Value PWTT0 and Preceding RR Interval Prr)

Referring again to FIG. 10, it is assumed that the first candidate value PWTT0 is the accurate PWTT. In this case, the first candidate value PWTT0 depends on the amount of blood stored in the heart during the preceding RR interval prr, as described in the second embodiment. That is, there is a negative correlation between the first candidate value PWTT0 and the preceding RR interval prr. On the other hand, assuming that the first candidate value PWTT0 is not the accurate PWTT, the first candidate value PWTT0 decreases as the preceding RR interval prr increases. That is, there is a strong negative correlation between the preceding RR interval prr and the first candidate value PWTT0. Therefore, in a case where the slope x0 calculated by the first calculator 325 is close to “−1” and the correlation coefficient y0 is close to “−1”, the specifying unit 327 can specify that the first candidate value PWTT0 is not the accurate PWTT.

### (Relationship Between Second Candidate Value PWTT1 and RR Interval Rr)

In a case where the second candidate value PWTT1 is the accurate PWTT, the second candidate value PWTT1 is an independent value and has no correlation with the RR interval rr. On the other hand, in a case where the second candidate value PWTT1 is not the accurate PWTT, the second candidate value PWTT1 increases as the RR interval rr increases. That is, in this case, there is a positive correlation between the second candidate value PWTT1 and the RR interval rr. Therefore, in a case where the slope x1 calculated by the second calculator 326 is close to “1” and the correlation coefficient y1 is close to “1”, the specifying unit 327 can specify that the second candidate value PWTT1 is not the accurate PWTT.

### Specific Example

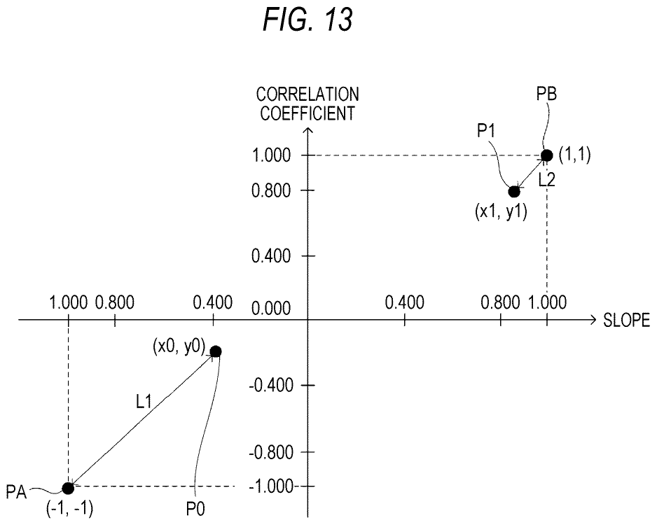

FIG. 13 illustrates an example of a relationship between the slope x0 and the correlation coefficient y0 calculated by the first calculator 325 of FIG. 9, and a relationship between the slope x1 and the correlation coefficient y1 calculated by the second calculator 326 of FIG. 9. In FIG. 13, a horizontal axis represents a slope, in which a vertical axis represents a correlation coefficient.

The specifying unit 327 is configured to calculate, for example, a distance L1 between a point P0 (x0, y0) and a point PA (−1, −1) plotted on the graph. The specifying unit 327 is configured to calculate, for example, a distance L2 between a point P1 (x1, y1) and a point PB (1, 1) plotted on the graph.

Then, as illustrated in FIG. 13, in a case where the distance L1 is larger than the distance L2 (L1>L2), the specifying unit 327 is configured to specify the first candidate value PWTT0 as the accurate PWTT. On the other hand, in a case where the distance L1 is smaller than the distance L2 (L1<L2), the specifying unit 327 is configured to specify the second candidate value PWTT1 as the accurate PWTT.

### Operations

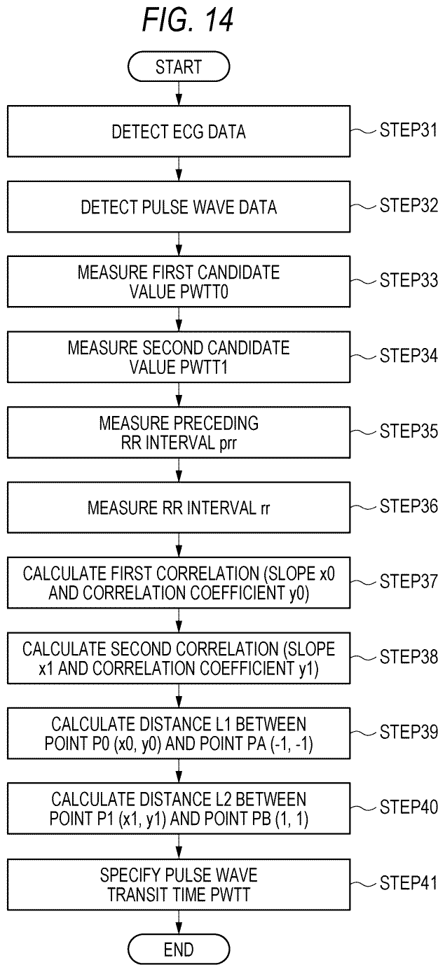

FIG. 14 is a flowchart illustrating operations in a case where the physiological information processing apparatus 300 according to the third embodiment specifies the PWTT of the subject.

Referring to FIG. 14, operations of STEP 31 and STEP 32 of FIG. 14 is the same as or similar to the operation of STEP 21 and STEP 22 of FIG. 8, and thus detailed description thereof will not be repeated here.

Next, the physiological information processing apparatus 300 sets one of a plurality of heartbeats as the reference time t20, and is configured to measure time from the reference time t20 to a rising timing of the pulse wave that is after and closest to the reference time t20 as the first candidate value PWTT0 (STEP 33).

Next, the physiological information processing apparatus 300 measures, as the second candidate value PWTT1, time from the reference time t20 to a rising timing of the pulse wave that is after the reference time t20 and next to the pulse wave closest to the reference time t20 (STEP 34).

Next, the physiological information processing apparatus 300 measures the preceding RR interval rpp that is time from the heartbeat before and closest to the reference time t20 to the reference time t20 (STEP 35).

Next, the physiological information processing apparatus 300 measures the RR interval rr that is time from the reference time t20 to the heartbeat that is after and closest to the reference time t20 (STEP 36).

Next, the physiological information processing apparatus 300 calculates the first correlation between the first candidate value PWTT0 and the preceding RR interval prr. Specifically, the physiological information processing apparatus 300 calculates, as the first correlation, the slope x0 and the correlation coefficient y0 of the regression line S1 in a case where a plurality of points are plotted with the preceding RR interval prr being the value of the horizontal axis and the first candidate value PWTT0 being the value of the vertical axis (STEP 37).

Next, the physiological information processing apparatus 300 calculates the second correlation between the second candidate value PWTT1 and the RR interval rr. Specifically, the physiological information processing apparatus 300 calculates, as the second correlation, the slope x1 and the correlation coefficient y1 of the regression line S2 in a case where a plurality of points are plotted with the RR interval rr being the value of the horizontal axis and the second candidate value PWTT1 being the value of the vertical axis (STEP 38).

Next, the physiological information processing apparatus 300 calculates the distance L1 between the point P0 (x0, y0) and the point PA (−1, −1) plotted on the graph (STEP S39).

Next, the physiological information processing apparatus 300 calculates the distance L2 between the point P1 (x1, y1) and the point PB (1, 1) plotted on the graph (STEP 40).

Next, the physiological information processing apparatus 300 specifies, as the PWTT of the subject, one of the first candidate value PWTT0 and the second candidate value PWTT1, based on the distance L1 and the distance L2. Specifically, in a case where the distance L1 is larger than the distance L2 (L1>L2), the physiological information processing apparatus 300 specifies the first candidate value PWTT0 as the accurate PWTT. On the other hand, in a case where the distance L1 is smaller than the distance L2 (L1<L2), the physiological information processing apparatus 300 specifies the second candidate value PWTT1 as the accurate PWTT (STEP 41).

### Modifications of Third Embodiment

(Description when Plural First Candidate Values PWTT0 are Divided into Plural Ranges)

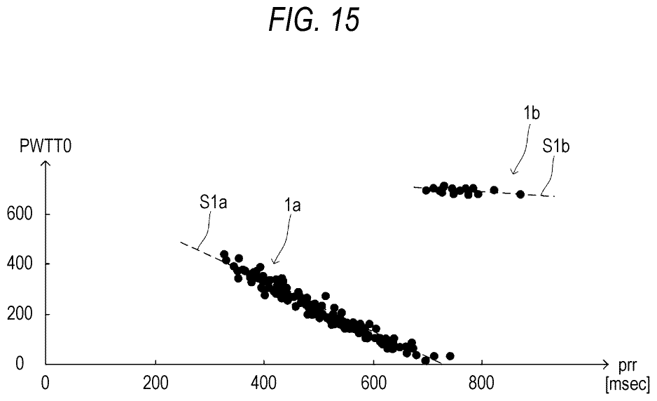

FIG. 15 illustrates a case where a plurality of first candidate values PWTT0 measured by the first candidate value measurement unit 321 of FIG. 9 are divided into a plurality of ranges. In the graph of FIG. 15, a horizontal axis represents a value of the preceding RR interval prr, and a vertical axis represents a value of the first candidate value PWTT0.

As illustrated in FIG. 15, in a case where a plurality of points are plotted with the first candidate value PWTT0 being the value of the vertical axis and the preceding RR interval prr being the value of the horizontal axis, the plurality of points may be divided into a plurality of groups. In the example illustrated in FIG. 15, the plurality of first candidate values PWTT0 are divided into a group of first candidate values PWTT0 included in a range from 0 to 500 (hereinafter, referred to as a “first candidate value group 1a”), and a group of first candidate values PWTT0 included in a range from 600 to 800 (hereinafter, referred to as a “first candidate value group 1b”).

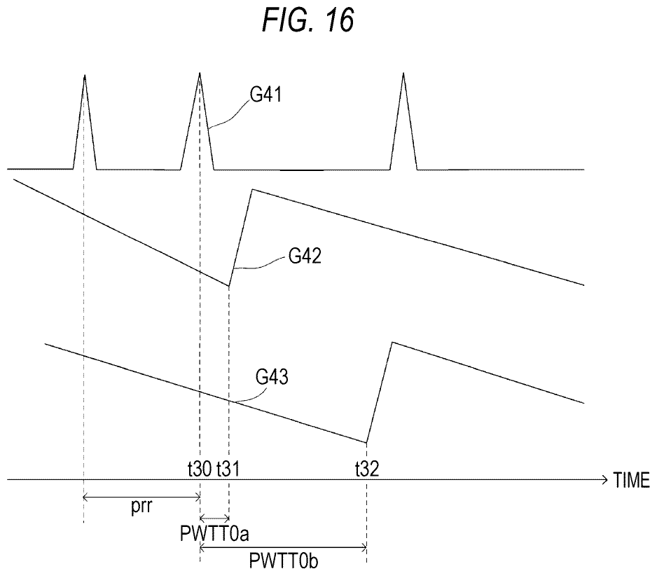

FIG. 16 is a graph of a relationship between a first candidate value PWTT0a in a case where the first candidate value group 1a in FIG. 15 is adopted, and a first candidate value PWTT0b in a case where the first candidate value group 1b in FIG. 15 is adopted. A graph G41 illustrates an ECG waveform. A graph G42 illustrates a photoplethysmogram waveform corresponding to the first candidate value group 1a, and a graph G43 illustrates a photoplethysmogram waveform corresponding to the first candidate value group 1b.

When it is assumed that the first candidate value group 1a is adopted with the reference time being a time point t30, time from the time point t30 to a time point t31 corresponds to the first candidate value PWTT0 in the graph G42. On the other hand, when it is assumed that the first candidate value group 1b is adopted, as illustrated in the graph G43, time from the time point t30 to a time point t32 corresponds to the first candidate value PWTT0.

In this manner, in a case where the plurality of first candidate values PWTT0 are divided by a range having a width of a prescribed value and are divided into a plurality of ranges, that is, in a case where the plurality of first candidate values PWTT0 are divided into a plurality of groups, it is necessary to appropriately select one of the plurality of groups.

(Description when Plural Second Candidate Values PWTT1 are Divided into Plural Ranges)

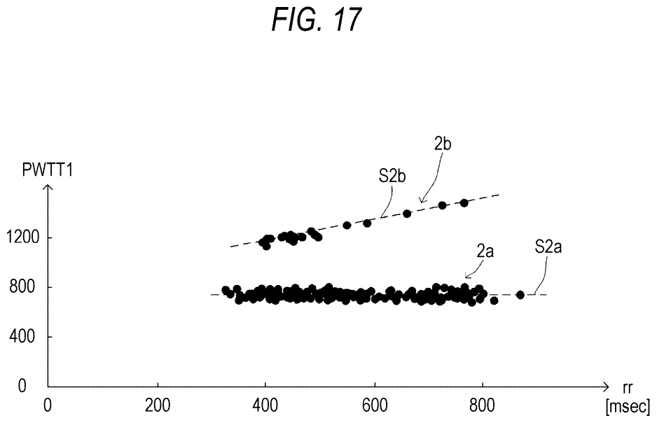

FIG. 17 illustrates a case where a plurality of second candidate values PWTT1 measured by the second candidate value measurement unit 323 of FIG. 9 are divided into a plurality of ranges. In the graph illustrated in FIG. 17, a horizontal axis represents a value of the RR interval rr, and a vertical axis represents a value of the second candidate value PWTT1.

As illustrated in FIG. 17, in a case where a plurality of points are plotted with the second candidate value PWTT1 being the value of the vertical axis and the RR interval rr being the value of the horizontal axis, the plurality of points may be divided into a plurality of groups and plotted. In the example illustrated in FIG. 17, the plurality of second candidate values PWTT1 are divided into a group of second candidate values PWTT1 included in a range from 600 to 900 (hereinafter, referred to as a “second candidate value group 2a”), and a group of second candidate values PWTT1 included in a range from 1000 to 1600 (hereinafter, referred to as a “second candidate value group 2b”).

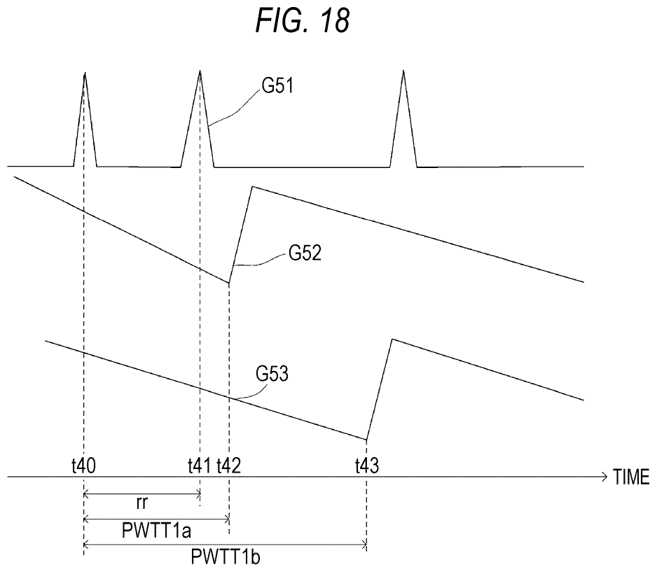

FIG. 18 is a graph illustrating a relationship between the second candidate value PWTT1a in a case where the second candidate value group 2a of FIG. 17 is adopted, and the second candidate value PWTT1b in a case where the second candidate value group 2b of FIG. 17 is adopted. A graph G51 illustrates an ECG waveform. A graph G52 illustrates a photoplethysmogram waveform corresponding to the second candidate value group 2a, and a graph G53 illustrates a photoplethysmogram corresponding to the second candidate value group 2b.

When it is assumed that the second candidate value group 2a is adopted with the reference time being a time point t40, time from the time point t40 to a time point t42 corresponds to the second candidate value PWTT1, as illustrated in the graph G52. On the other hand, when it is assumed that the second candidate value group 2b is adopted, as illustrated in the graph G53, time from the time point t40 to a time point t43 corresponds to the second candidate value PWTT1.

As described above, in a case where the plurality of second candidate values PWTT1 are divided by a range having a width of a prescribed value and are divided into a plurality of ranges, that is, in a case where the plurality of second candidate values PWTT1 are divided into a plurality of groups, it is necessary to appropriately select one of the plurality of groups.

### (Group Selection Method)

Here, a case where a plurality of first candidate values PWTT0 are divided into a plurality of groups as illustrated in FIG. 15, and a plurality of second candidate values PWTT1 are divided into a plurality of groups as illustrated in FIG. 17 will be described.

In such a case, as illustrated in FIG. 15, the first candidate value measurement unit 321 is configured to calculate, as the first correlation, the slope x0 and the correlation coefficient y0 for each of a regression line Sla corresponding to the first candidate value group 1a, and a regression line S1b corresponding to the first candidate value group 1b. The slope x0 and the correlation coefficient y0 for the regression line Sla are referred to as a slope x0a and a correlation coefficient y0a, respectively. The slope x0 and the correlation coefficient y0 for the regression line S1b are referred to as a slope x0b and a correlation coefficient y0b, respectively.

As illustrated in FIG. 17, the second candidate value measurement unit 323 is configured to calculate, as the second correlation, the slope x1 and the correlation coefficient y1 for each of a regression line S2a corresponding to the second candidate value group 2a, and a regression line S2b corresponding to the second candidate value group 2b. The slope x1 and the correlation coefficient y1 for the regression line S2a are referred to as a slope x1a and a correlation coefficient y1a, respectively. The slope x1 and the correlation coefficient y1 for the regression line S2b are referred to as a slope x1b and a correlation coefficient y1b, respectively.

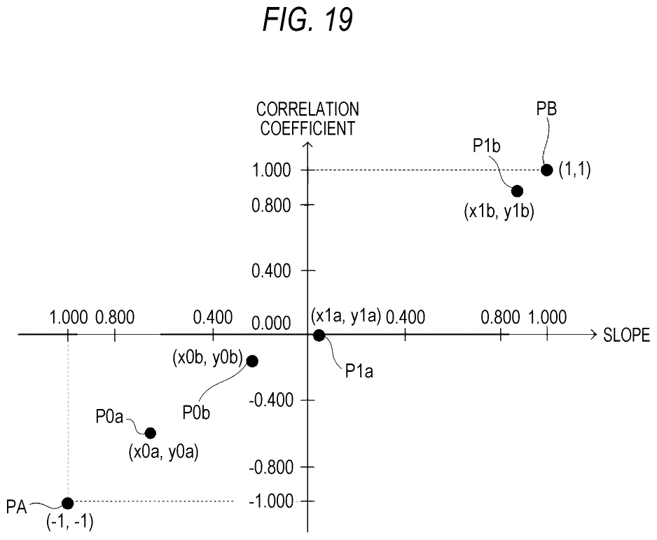

FIG. 19 illustrates an example of a relationship between the slopes x0a and x0b and the correlation coefficients y0a and y0b calculated by the first calculator 325 of FIG. 9, and the slopes x1a and x1b and the correlation coefficients y1a and y1b calculated by the second calculator 326 of FIG. 9. In FIG. 19, a horizontal axis indicates the slope, and a vertical axis indicates the correlation coefficient.

Regarding the first candidate value PWTT0, for example, as illustrated in FIG. 19, the specifying unit 327 is configured to plot a point P0a (x0a, y0b) and a point P0b (x0b, y0b) on a graph, and is configured to specify one of the point P0a and the point P0b that is farther from the point PA (−1, −1) that is a first prescribed value.

That is, in a case where the point P0b is farther from the point PA, the specifying unit 327 is configured to select the first candidate value group 1b, as a group to be adopted from the first candidate value group 1a and the first candidate value group 1b of FIG. 15. On the other hand, in a case where the point P0a is farther from the point PA, the specifying unit 327 is configured to select the first candidate value group 1a, as a group to be adopted from the first candidate value group 1a and the first candidate value group 1b. Then, based on the plurality of first candidate values PWTT0 included in the selected group, the specifying unit 327 is configured to specify the PWTT of the subject using the method described in the third embodiment.

For example, as illustrated in FIG. 19, the specifying unit 327 is configured to plot a point P1a (x1a, y1b) and a point P1b (x1b, y1b) on a graph relative to the second candidate value PWTT1, and is configured to specify one of the point P1a and the point P1b that is farther from the point PB (1, 1) that is a second prescribed value.

That is, in a case where the point P1b is farther from the point PB, the specifying unit 327 is configured to select the second candidate value group 2b, as a group to be adopted from the second candidate value group 2a and the second candidate value group 2b of FIG. 17. On the other hand, in a case where the point P1a is farther from the point PB, the specifying unit 327 is configured to select the second candidate value group 2a, as a group to be adopted from the second candidate value group 2a and the second candidate value group 2b. Then, based on the plurality of second candidate values PWTT1 included in the selected group, the specifying unit 327 is configured to specify the PWTT of the subject using the method described in the third embodiment.

The specifying unit 327 may be configured to select a group to be adopted by another selection method instead of being limited to the above-described selection method. For example, the specifying unit 327 may be configured to calculate an average value of a plurality of first candidate values PWTT0 included in the first candidate value group 1a and an average value of a plurality of first candidate values PWTT0 included in the first candidate value group 1b, and configured to adopt a group corresponding to the larger average value, or may be configured to calculate an average value of a plurality of second candidate values PWTT1 included in the second candidate value group 2a and an average value of a plurality of second candidate values PWTT1 included in the second candidate value group 2b, and configured to adopt a group corresponding to the smaller average value.

In the physiological information processing apparatus 300 according to the third aspect of the present disclosure as described above, the first candidate value measurement unit 321 is configured to set one of a plurality of heartbeats as the reference time t20, and is configured to measure, as the first candidate value PWTT0, the time from the reference time t20 to the rising timing t21 of the pulse wave that is after and closest the reference time t20. The second candidate value measurement unit 323 is configured to measure, as the second candidate value PWTT1, the time from the reference time t20 to the rising timing t23 of the pulse wave that is after the reference time t20 and next to the pulse wave closest to the reference time t20. The preceding RR interval measurement unit 322 is configured to measure the preceding RR interval prr that is time from a heartbeat before and closest to the reference time t20 to the reference time t20. The RR interval measurement unit 324 is configured to measure the RR interval rr that is the time from the reference time t20 to the heartbeat that is after and closest to the reference time t20. The specifying unit 327 is configured to specify the PWTT of the subject, based on the first candidate value PWTT0, the second candidate value PWTT1, the preceding RR interval prr, and the RR interval rr.

Here, in a case where the first candidate value PWTT0 is the correct PWTT, there is a negative correlation between the first candidate value PWTT0 and the preceding RR interval rpp. On the other hand, in a case where the first candidate value PWTT0 is not the correct PWTT, there is a strong negative correlation between the first candidate value PWTT0 and the preceding RR interval rpp.

In a case where the second candidate value PWTT1 is the correct PWTT, there is no correlation between the second candidate value PWTT1 and the RR interval rr. On the other hand, in a case where the second candidate value PWTT1 is not the correct PWTT, there is a strong positive correlation between the second candidate value PWTT1 and the RR interval rr.

Therefore, the PWTT of the subject can be more accurately measured by the first candidate value PWTT0, the second candidate value PWTT1, the preceding RR interval rpp, and the RR interval rr as described above.

In the physiological information processing apparatus 300 according to the third aspect of the present disclosure as described above, the first calculator 325 is configured to calculate the first correlation between the first candidate value PWTT0 and the preceding RR interval prr. The second calculator 326 is configured to calculate the second correlation between the second candidate value PWTT1 and the RR interval rr. Based on the first correlation and the second correlation, the specifying unit 327 is configured to specify one of the first candidate value PWTT0 and the second candidate value PWTT1, as the PWTT of the subject.

With this configuration, the correct PWTT can be more reliably specified between the first candidate value PWTT0 and the second candidate value PWTT1.

In the physiological information processing apparatus 300 according to the third aspect of the present disclosure as described above, the first candidate value measurement unit 321 is configured to measure a plurality of first candidate values PWTT0, based on the ECG data and the pulse wave data in a prescribed period. The preceding RR interval measurement unit 322 is configured to measure a plurality of preceding RR intervals prr, based on the ECG data and the pulse wave data in a prescribed period. The first calculator 325 is configured to calculate the first correlation coefficient between the plurality of first candidate values PWTT0 and the plurality of preceding RR intervals prr. In a case where the plurality of first candidate values PWTT0 are divided by a range having a width of a prescribed value and are divided into a plurality of ranges, the first calculator 325 is configured to calculate the first correlation coefficient for each range. In a case where a plurality of first correlation coefficients are calculated, the specifying unit 327 is configured to specify the PWTT of the subject, based on the first correlation coefficient having a value farthest from the first prescribed value among the plurality of first correlation coefficients.

With such a configuration, for example, even in a case where the plurality of first candidate values PWTT0 are divided into a plurality of ranges having largely different values, the first correlation coefficient can be calculated for each range and a more appropriate first correlation coefficient can be used.

In the physiological information processing apparatus 300 according to the third aspect of the present disclosure as described above, the second candidate value measurement unit 323 is configured to measure a plurality of second candidate values PWTT1, based on the ECG data and the pulse wave data in a prescribed period. The RR interval measurement unit 324 is configured to measure a plurality of RR intervals rr, based on the ECG data and the pulse wave data in a prescribed period. The second calculator 326 is configured to calculate the second correlation coefficient between the plurality of second candidate values PWTT1 and the plurality of RR intervals rr. In a case where the plurality of second candidate values PWTT1 are divided by a range having a width of a prescribed value and are divided into a plurality of ranges, the second calculator 326 is configured to calculate the second correlation coefficient for each range. In a case where a plurality of second correlation coefficients are calculated, the specifying unit 327 is configured to specify the PWTT of the subject, based on the second correlation coefficient having a value farthest from the second prescribed value among the plurality of second correlation coefficients.

With such a configuration, for example, even in a case where the plurality of second candidate values PWTT1 are divided into a plurality of ranges having largely different values, the second correlation coefficient can be calculated for each range and a more appropriate second correlation coefficient can be used.

Although the embodiments of the present disclosure have been described above, the technical scope of the present disclosure should not be construed as being limited to the description of the present embodiments. The present embodiments are merely examples, and it is understood by those skilled in the art that various modifications of the embodiments are possible within the scope of the present disclosure described in the claims. The technical scope of the present application should be determined based on the scope of the inventions described in the claims and equivalents thereof.

This application claims priority to Japanese Patent Application No. 2023-023465 filed on Feb. 17, 2023, the entire content of which is incorporated herein by reference.

### INDUSTRIAL APPLICABILITY

According to the present disclosure, it is possible to measure pulse wave transit time of a subject more accurately.

1. A physiological information processing apparatus comprising:
a controller configured to:
detect ECG data of a subject;
detect pulse wave data of the subject; and
configured to measure pulse wave transit time of the subject, based on the ECG data and the pulse wave data,
wherein the controller is configured to:
set one of rising timings of pulse waves as reference time, and configured to measure, as candidate values of the pulse wave transit time, time from the heartbeats to the reference time for a plurality of heartbeats before the reference time; and
specify, as the pulse wave transit time of the subject, the candidate value satisfying a prescribed condition, among the candidate values.

2. The physiological information processing apparatus according to claim 1,
wherein the controller is configured to specify, as the pulse wave transit time of the subject, a smallest candidate value that is equal to or greater than a lower limit value of a set standard range of the pulse wave transit time, among the candidate values.

3. The physiological information processing apparatus according to claim 1,
wherein the controller is configured to specify, as the pulse wave transit time of the subject, the candidate value that is equal to or greater than a lower limit value of a set standard range of the pulse wave transit time and that is less than an upper limit value of the standard range, among the candidate values.

4. The physiological information processing apparatus according to claim 2,
wherein the controller is further configured to set the standard range, based on an attribute of the subject.

5. The physiological information processing apparatus according to claim 1,
wherein the controller is further configured to measure an RR interval that is time from each heartbeat before the reference time to a heartbeat before and closest to the reference time, and
the controller is configured to specify the pulse wave transit time of the subject, based on a correlation between the candidate value and the RR interval.

6. The physiological information processing apparatus according to claim 1,
wherein the controller is further configured to measure a preceding RR interval that is time from an immediately preceding heartbeat of each heartbeat before the reference time to the each heartbeat before the reference time, and
the controller is configured to specify the pulse wave transit time of the subject, based on a correlation between the candidate value and the preceding RR interval.

7. A physiological information processing apparatus comprising:
a controller configured to:
detect ECG data of a subject;
detect pulse wave data of the subject; and
measure pulse wave transit time of the subject, based on the ECG data and the pulse wave data,
wherein the controller is configured to:
set one of a plurality of heartbeats as reference time, and configured to measure time, as a first candidate value, from the reference time to a rising timing of a pulse wave after and closest to the reference time;
measure time, as a second candidate value, from the reference time to a rising timing of a pulse wave next to the pulse wave after and closest to the reference time;
measure a preceding RR interval that is time from a heartbeat before and closest to the reference time to the reference time;
measure an RR interval that is time from the reference time to a heartbeat after and closest to the reference time; and
specify the pulse wave transit time of the subject, based on the first candidate value, the second candidate value, the preceding RR interval, and the RR interval.

8. The physiological information processing apparatus according to claim 7,
wherein the controller is further configured to:
calculate a first correlation between the first candidate value and the preceding RR interval; and
calculate a second correlation between the second candidate value and the RR interval, and
the controller is configured to specify, as the pulse wave transit time of the subject, one of the first candidate value and the second candidate value, based on the first correlation and the second correlation.

9. The physiological information processing apparatus according to claim 8,
wherein the controller is configured to:
measure a plurality of the first candidate values, based on the ECG data and the pulse wave data in a prescribed period,
measure a plurality of the preceding RR intervals, based on the ECG data and the pulse wave data in the prescribed period;
calculate a first correlation coefficient between the plurality of first candidate values and the plurality of preceding RR intervals;
in a case where the plurality of first candidate values are divided by a range having a width of a prescribed value and are divided into a plurality of ranges, calculate the first correlation coefficient for each of the ranges; and
in a case where a plurality of the first correlation coefficients are calculated, specify the pulse wave transit time of the subject, based on the first correlation coefficient having a value farthest from the first prescribed value among the plurality of first correlation coefficients.

10. The physiological information processing apparatus according to claim 8,
wherein the controller is configured to:
measure a plurality of the second candidate values, based on the ECG data and the pulse wave data in a prescribed period;
measure a plurality of the RR intervals, based on the ECG data and the pulse wave data in the prescribed period;
calculate a second correlation coefficient between the plurality of second candidate values and the plurality of RR intervals;
in a case where the plurality of second candidate values are divided by a range having a width of a prescribed value and are divided into a plurality of ranges, calculate the second correlation coefficient for each of the ranges; and
in a case where a plurality of the second correlation coefficients are calculated, specify the pulse wave transit time of the subject, based on the second correlation coefficient having a value farthest from the second prescribed value among the plurality of second correlation coefficients.

11. A physiological information processing method executed by the physiological information processing apparatus according to claim 1, the physiological information processing method comprising:
detecting ECG data of a subject;
detecting pulse wave data of the subject; and
measuring pulse wave transit time of the subject, based on the ECG data and the pulse wave data,
wherein the measuring of the pulse wave transit time of the subject includes:
setting one of rising timings of pulse waves as reference time, and
measuring, as candidate values of the pulse wave transit time, time from the heartbeats to the reference time for a plurality of heartbeats before the reference time; and
specifying, as the pulse wave transit time of the subject, the candidate value satisfying a prescribed condition among the candidate values.

12. A physiological information processing method executed by the physiological information processing apparatus according to claim 7, the physiological information processing method comprising:
detecting ECG data of a subject;
detecting pulse wave data of the subject; and
measuring pulse wave transit time of the subject, based on the ECG data and the pulse wave data,
wherein the measuring of the pulse wave transit time of the subject includes:
setting one of a plurality of heartbeats as reference time and measuring time, as a first candidate value, from the reference time to a rising timing of a pulse wave after and closest to the reference time;
measuring time, as a second candidate value, from the reference time to a rising timing of a pulse wave next to the pulse wave after and closest to the reference time;
measuring a preceding RR interval that is time from a heartbeat before and closest to the reference time to the reference time;
measuring an RR interval that is time from the reference time to a heartbeat after and closest to the reference time; and
specifying the pulse wave transit time of the subject, based on the first candidate value, the second candidate value, the preceding RR interval, and the RR interval.

13. A non-transitory computer readable storage medium storing a physiological information processing program used for a physiological information processing apparatus, the program comprising instructions which, when executed by a computer, cause the computer in the physiological information processing apparatus to:
detect ECG data of a subject;
detect pulse wave data of the subject; and
measure pulse wave transit time of the subject, based on the ECG data and the pulse wave data,
wherein the measuring of the pulse wave transit time of the subject includes:
setting one of rising timings of pulse waves as reference time, and measuring, as candidate values of the pulse wave transit time, time from the heartbeats to the reference time for a plurality of heartbeats before the reference time; and
specifying, as the pulse wave transit time of the subject, the candidate value satisfying a prescribed condition among the candidate values.

14. A non-transitory computer readable storage medium storing a physiological information processing program used for a physiological information processing apparatus, the program comprising instructions which, when executed by a computer, cause the computer in the physiological information processing apparatus to:
detect ECG data of a subject;
detect pulse wave data of the subject; and
measure pulse wave transit time of the subject, based on the ECG data and the pulse wave data,
wherein the measuring of the pulse wave transit time of the subject includes:
setting one of a plurality of heartbeats as reference time and measuring time, as a first candidate value, from the reference time to a rising timing of a pulse wave after and closest to the reference time;
measuring time, as a second candidate value, from the reference time to a rising timing of a pulse wave next to the pulse wave after and closest to the reference time;
measuring a preceding RR interval that is time from a heartbeat before and closest to the reference time to the reference time;
measuring an RR interval that is time from the reference time to a heartbeat after and closest to the reference time; and
specifying the pulse wave transit time of the subject, based on the first candidate value, the second candidate value, the preceding RR interval, and the RR interval.
